(19) United States
(12) Patent Application Publication
(10) Pub. No.: US 2006/0275341 A1
(43) Pub. Date: Dec. 7, 2006

(54) Thin foam coating comprising discrete, closed-cell capsules

(75) Inventors: Dean-Mo Liu, Richmond (CA); Mao-Jung Maurice Lien, Maple Ridge (CA); Doug Smith, Vancouver (CA)

Correspondence Address: OYEN, WIGGS, GREEN & MUTALA LLP;480 - THE STATION, 601 WEST CORDOVA STREET, VANCOUVER, BC, V6B 1G1, CA

(73) Assignee: MIV Therapeutics Inc., Vancouver (CA)

(21) Appl. No.: 11/142,500

(22) Filed: Jun. 2, 2005

(51) Int. Cl. A61F 2/02

(52) U.S. Cl. 424/426

(57) ABSTRACT

This application relates to a thin foam coating comprising discrete, closed-cell capsules. The coating may be applied to an implantable medical device, such as a stent. The closed-cell capsules each having an outer polymeric shell and an inner liquid core containing the drug. The polymeric shells degrade in vivo to achieve controlled elution of the drug.

### TECHNICAL FIELD

This application relates to coatings for implantable medical devices for drug delivery purposes.

### BACKGROUND

Drug-coated medical devices are well known in the prior art. For example, drug-eluting intravascular stents have been shown to improve overall therapeutic performance after implantation or deployment of the coated stent within the lesion of a blood vessel. Drugs such as paclitaxel are typically employed to reduce restenosis at the site of implantation.

In order to be effective, drug-eluting stents are engineered to carry and release drugs in a controlled manner. Conventional approaches involve incorporating a therapeutic drug in a polymer solution, then coating the stent with the polymer. Drug can then be released over a period of time after deployment in vivo. U.S. Pat. No. 6,585,764 entitled “Stent with therapeutically active dosage of rapamycin coated thereon” describes delivery of rapamycin drug using a polymer matrix as a drug carrier. The polymer includes both degradable and non-degradable components. The drug-polymer mixture is coated via spraying or dipping on to a stent to achieve controlled release of the drug.

Co-pending U.S. patent application No. 60/636,105 filed 16 Dec. 2004, which is hereby incorporated by reference, describes a multi-layer drug delivery device and method of manufacturing same. The device includes at least one first layer containing a drug and at least one second layer comprising a polymer for regulating release of the drug. For example, the second layer is preferably biodegradable, bioabsorbable and/or bioresolvable in vivo to permit gradual exposure of the first layer and elution of the drug therefrom. The first and second layers are formulated using immiscible solvents to substantially prevent inter-diffusion between the drug and polymer layers.

The present invention employs a modified approach to achieve regulated elution of drugs from implanted medical devices. In the present invention the drug is deployed in a foam comprising a plurality of discrete closed-cell capsules rather than in a uniform layer.

### SUMMARY OF INVENTION

In accordance with the invention, a drug delivery device is disclosed comprising a substrate and at least one layer of drug-containing emulsified foam applied to the substrate. The foam comprises a plurality of discrete closed-cell capsules each having an outer polymeric shell and an inner core containing the drug.

A method of manufacturing a drug delivery device is also described comprising providing a substrate; providing a first solution comprising a drug dissolved in one or more first solvents; providing a second solution comprising a polymer dissolved in one or more second solvents; combining the first solution and the second solution to form an emulsified solution comprising a plurality of closed-cell capsules each having an outer polymeric shell and an inner core containing the drug; applying at least one coating of said emulsified solution to the substrate; and removing the second solvent from the emulsified solution to form at least one thin layer of emulsified foam on the substrate, the foam comprising the closed-cell capsules.

The application also describes the use of the device to deliver drugs to a target location, such as the site of a blood vessel lesion in vivo.

### BRIEF DESCRIPTION OF DRAWINGS

In drawings which illustrate embodiments of the invention, but which should not be construed as restricting the spirit or scope of the invention in any way,

FIG. 1 is a schematic view of an implantable medical device having a thin foam coating applied thereto.

FIGS. 2 is a scanning electron microscopy (SEM) photograph showing a cross-section of a closed-cell thin foam formulated in accordance with the invention.

FIG. 3 is a SEM photograph showing a top view of a closed-cell thin foam formulated in accordance with the invention

FIG. 4 is graph showing a representative elution profile for a drug deployed in accordance with the invention

### DESCRIPTION

Throughout the following description, specific details are set forth in order to provide a more thorough understanding of the invention. However, the invention may be practiced without these particulars. In other instances, well known elements have not been shown or described in detail to avoid unnecessarily obscuring the invention. Accordingly, the specification and drawings are to be regarded in an illustrative, rather than a restrictive, sense.

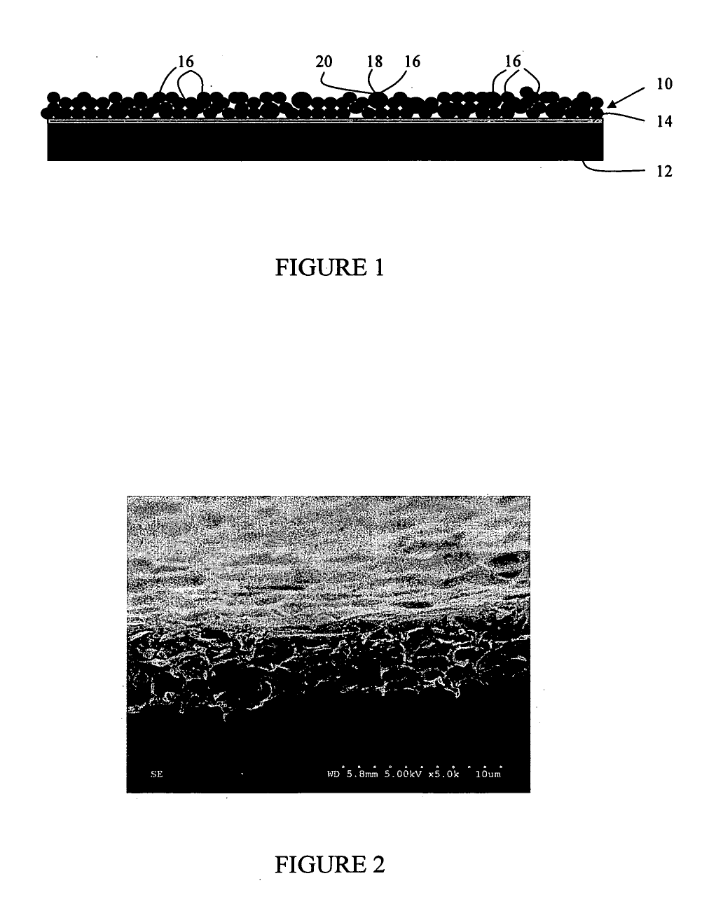

This application describes the structure and synthesis of a thin foam coating 10 which may be applied to an implantable medical device 12 for drug delivery purpose. As shown in FIG. 1, medical device 12 may have a bicompatible layer 14 applied to its outer surface for receiving coating 10. For example, biocompatible layer 14 may comprise an oxide layer applied to the outer surface of substrate 12. The oxide layer may be formed, for example, by thermal or chemical means. As will be apparent to a person skilled in the art, various means for surface modification may be employed, such as the method employed in Applicant's co-pending Patent Cooperation Treaty application No. PCT/CA2004/001585 which is hereby incorporated by reference.

Although the present invention is described in relation to metal substrates such as implantable medical devices, the invention may be useful in other applications where it is desirable to deliver a drug to a target site. The invention may have application, for example, for medical devices which are not permanently implanted in vivo or medical devices used in peripheral rather than coronary applications. Further, substrate 12 may be a non-metal, such as a ceramic, polymeric or composite material.

As shown in FIG. 1, coating 10 is a thin foam comprised of a plurality of closed-cell capsules 16. Each capsule 16 includes an inner core 18 containing the drug or therapeutically active agent and an outer polymeric shell 20. Coating 10 may comprise multiple layers of capsules 16. As described below, the outermost layers of capsules 16 may gradually degrade in vivo to elute the drug encapsulated therein. Capsules 16 may range in size from about 10 nm to about 5,000 nm in diameter. By way of illustration, FIG. 2 shows a cross-sectional view of a coating 10 having a thickness of approximately 5 μm consisting of approximately 4-5 layers of capsules 16. In this example, each layer is approximately 1-2 μm in size. The polymeric shells 20 separating the discrete drug-containing cores 18 are formed of poly(lactic-co-glycolic acid) (PLGA) in this example.

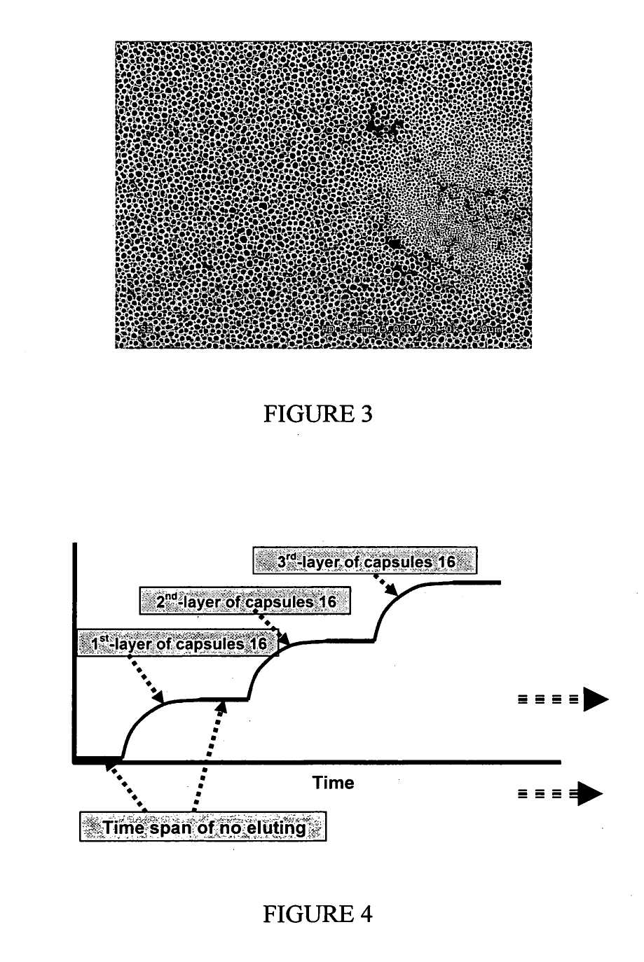

FIG. 3 shows a top view of a coating 10 wherein the polymeric shells 20 encapsulating capsules 16 have a thickness of approximately 0.2-5 μm in size. Again, shells 20 are formed from PLGA in this example.

In one embodiment of the invention the drug-containing inner core 18 of each capsule 16 is a liquid derived from a first solution comprising a drug or other therapeutically active agent dissolved in one or more hydrophilic solvents. In one embodiment the liquid inner core 18 may in the form of a paste. The drug within core 18 may be poorly soluble or insoluble in water, such as paclitaxel. Alternatively, the drug may be water soluble. The hydrophilic solvents may comprise a mixture of solvents selected from, but not limited to, ethylene glycol, propylene glycol, glycerin, DMSO, DENA, Cremorphor, and water.

The polymeric shell 20 of each capsule 16 is derived from a second solution of a biocompatible and biodegradable polymer dissolved in one or more hydrophobic solvents. By way of example, the polymer may include polylactide, polyglycolide, poly(lactide-co-glycolide), polycaprolactone, polysulfone, polyurethane, ethylene vinyl-acetate and mixtures thereof. The hydrophobic solvent may include, for example, chloroform, methylene dichloride, methylene trichloride, ethylene dichloride, ethylene acetate, butyl acetate, hexanes, heptanes and mixtures containing two or more of the preceding solvents.

The first, drug-containing solution is distributed and suspended in the second, polymer solution to form a stable emulsified solution. The drug-containing phase is distributed homogeneously in the polymer by conventional means known in the art such as emulsification, homogenization, ultrasonication, and atomization. Preferably coating 10 is formulated to avoid interaction between the discrete emulsified phase and the continuous polymer phase. That is, there is no inter- or cross-diffusion between the drug dissolved in the hydrophilic first solution and the hydrophobic polymer second solution.

The emulsified solution may be coated on to the biocompatible layer 14 of substrate 12 (FIG. 1). For example, substrate 12 may be an implantable medical device, such as a stent. As indicated above, substrate 12 may be formed of various different materials, such as metals, ceramics, polymers or composites, and surface treatment of substrate 12 to enhance biocompatibility or to enhance coating coverage is optional. As will be appreciated by a person skilled in the art, the emulsified solution may be applied to substrate 12 by various means including spraying, dipping, brushing, and printing to form a thin coating 10. Once coating 10 is applied, the hydrophobic solvent may be rapidly removed by natural or forced evaporation, resulting in layers of discrete, tiny capsules 16 (FIG. 1) upon drying. The resulting thin foam coating 10 contains both the drug-containing liquid phase in the inner cores of 18 of discrete capsules 16 and the polymer solid phase in the outer shells 20 of capsules 16. In one embodiment, the concentration of the drug within the capsule inner cores 18 comprises between 0.01 to 70% of coating 10 by weight, or more particularly between 0.1 to 50% by weight. The polymeric shell 20 may comprise between 30 and 99.9% of coating 10 by weight, or more particularly between 50 and 99.5% by weight. If the concentration of the polymer in coating 10 is less than about 30% by weight, this may result in structural disintegrity of the resulting thin foam coating 10. This may in turn weaken the adhesion of coating 10 to substrate 12.

In use, a coated medical device having the structure illustrated in FIG. 1 may be implanted in vivo. The layered, closed-cell structure of capsules 16 achieves a slow and step-wise drug release profile, as schematically illustrated in FIG. 4. In this example, the outermost layer of capsules 16 releases drug as the outermost polymeric shells 20 degrade. This causes gradual elution of drug from capsule inner cores 18. The drug may be released either by diffusion through the polymer walls or by direct release if the polymer walls burst. The invention is especially effective in achieving controlled release of poorly water-soluble or water-insoluble drugs, such as paclitaxel, into blood or tissue at the target location in vivo.

As shown in FIG. 4, the initial phase of drug elution may be followed by a time span of no elution during which the second layer of capsules 16 begins to degrade. Once the degradation has progressed to a threshold extent, then elution of the drug will once again commence. As shown in FIG. 4, the same degradation-release scenario may take place in a layer by layer fashion until the thin coating 10 is completely degraded. The timing and profile of drug release can be easily adjusted by altering the type and thickness of polymer, for example to lengthen the total time span of drug release from days to weeks or months. As will be appreciated by a person skilled in the art, coating 10 may also be configured so that different types of drugs or other therapeutic agents may be released, either simultaneously or sequentially. Further, in another embodiment of the invention, capsules 16 could be arranged so that drug is released continuously at a substantially constant rate rather than in a step-wise fashion.

As will be apparent to those skilled in the art in the light of the foregoing disclosure, many alterations and modifications are possible in the practice of this invention without departing from the spirit or scope thereof. Accordingly, the scope of the invention is to be construed in accordance with the substance defined by the following claims.

What is claimed is:

1. A drug delivery device comprising:
(a) a substrate;
(b) at least one layer of drug-containing emulsified foam applied to said substrate, wherein said foam comprises a plurality of discrete closed-cell capsules each having an outer polymeric shell and an inner core containing said drug.

2. The drug delivery device as defined in claim 1, wherein there is no interdiffusion of said drug between said inner core and said polymeric shell.

3. The drug delivery device as defined in claim 1, wherein said capsules are each between 10 and 5,000 nm in diameter.

4. The drug delivery device as defined in claim 3, wherein the thickness of said outer polymeric shell is between 0.1-5 μm in size.

5. The drug delivery device as defined in claim 1, wherein said plurality of discrete closed-cell capsules independently release said drug.

6. The drug delivery device as defined in claim 1, wherein said inner core is in a liquid phase.

7. The drug delivery device as defined in claim 6, wherein said drug is insoluble or poorly soluble in water.

8. The drug delivery device as defined in claim 6, wherein said drug is water soluble.

9. The drug delivery device as defined in claim 1, wherein said device comprises a plurality of layers of said emulsified foam.

10. The drug delivery device as defined in claim 9, wherein each of said layers has a thickness less than 5 μm in size.

11. The drug delivery device as defined in claim 9, wherein said layers are arranged so that said drug is released from said device in a step-wise manner as said polymeric shell of said capsules is gradually degraded.

12. The drug delivery device as defined in claim 11, wherein said drug is released in a dissolved form.

13. The drug delivery device as defined in claim 9, wherein different ones of said layers of said device contain different drugs.

14. The drug delivery device as defined in claim 1, wherein the concentration of said drug in each of said capsules is between 0.01 to 70% by weight.

15. The drug delivery device as defined in claim 14, wherein the concentration of said drug in each of said capsules is between 0.1 to 50% by weight.

16. The drug delivery device as defined in claim 1, wherein said polymeric shell comprises between 30 and 99.9% of said foam by weight.

17. The drug delivery device as defined in claim 16, wherein said polymeric shell comprises between 50 to 99.5% of said foam by weight.

18. The drug delivery device as defined in claim 1, wherein said polymeric shell is biocompatible.

19. The drug delivery device as defined in claim 18, wherein said polymeric shell is formed from material selected from the group consisting of polylactide, polyglycolide, poly(lactide-co-glycolide), polycaprolactone, polysulfone, polyurethane, ethylene vinyl-acetate and mixtures thereof.

20. The drug delivery device as defined in claim 1, wherein said substrate is formed from a material selected from the group consisting of metal, ceramic, polymer and composites thereof.

21. The drug delivery device as defined in claim 20, wherein said substrate is an implantable medical device.

22. The drug delivery device as defined in claim 21, wherein said implantable medical device is a stent.

23. The drug delivery device as defined in claim 21, wherein said foam is applied to a biocompatible outer surface of said medical device.

24. A method of manufacturing a drug delivery device comprising:
(a) providing a substrate;
(b) providing a first solution comprising a drug dissolved in one or more first solvents;
(c) providing a second solution comprising a polymer dissolved in one or more second solvents;
(d) combining said first solution and said second solution to form an emulsified solution comprising a plurality of closed-cell capsules each having an outer polymeric shell and an inner core containing said drug;
(e) applying at least one coating of said emulsified solution to said substrate; and
(f) removing said second solvent from said emulsified solution to form at least one thin layer of emulsified foam on said substrate, said foam comprising said closed-cell capsules.

25. The method as defined in claim 24, wherein said inner core containing said drug is in a liquid phase.

26. The method as defined in claim 24, wherein said first solvent is hydrophilic and said second solvent is hydrophobic.

27. The method as defined in claim 24, wherein said first solvent is hydrophobic and said second solvent is hydrophilic.

28. The method as defined in claim 24, wherein said capsules are distributed substantially homogeneously throughout said emulsified solution and said emulsified foam.

29. The method as defined in claim 24, comprising applying multiple coatings of said emulsified solution to said substrate to form multiple layers of said foam.

30. The method as defined in claim 29, wherein each of said coatings is applied in a thin film such that said layers each has a thickness less than 5 μm in size.

31. The method as defined in claim 30, wherein said coatings are applied such that said drug is released from said layers in a step-wise manner as said polymeric shells are gradually degraded.

32. The method as defined in claim 29, wherein different ones of said coatings and said layers derived therefrom contain different drugs.

33. The method as defined in claim 24, wherein said one or more first solvents is selected from the group consisting of ethylene glycol, propylene glycol, glycerol, glycerin, Cremorphor, DMSO, DENA, water and mixtures containing two or more of the preceding solvents.

34. The method as defined in claim 24, wherein said one or more second solvents is selected from the group consisting of chloroform, methylene dichloride, methylene trichloride, ethylene dichloride, ethylene acetate, butyl acetate, hexanes, heptanes and mixtures containing two or more of the preceding solvents.

35. The method as defined in claim 24, wherein said one or more second solvents is selected from the group consisting of polylactide, polyglycolide, poly(lactide-co-glycolide), polycaprolactone, polysulfone, polyurethanes, ethylene vinyl-acetate and mixtures containing two or more of the preceding solvents.

36. The method as defined in, claim 24, wherein the concentration of said drug in each of said capsules is between 0.01 to 70% by weight.

37. The method as defined in claim 36, wherein the concentration of said drug in each of said capsules is between 0.1 to 50% by weight.

38. The method as defined in claim 27, wherein polymeric shell comprises between 30 and 99.9% of said foam by weight.

39. The method as defined in claim 38, wherein said polymeric shell comprises between 50 to 99.5% of said foam by weight.

40. The method as defined in claim 24, wherein said second solvent is removed by evaporation

41. The method as defined in claim 24, comprising treating said substrate prior to application of said emulsified solution to improve the surface coverage of said coating thereon.

42. The method as defined in claim 24, wherein said coating is applied by a process selected from the group consisting of spraying, dipping, brushing and printing.

43. The use of a drug delivery device as defined in claim 1, wherein said use comprises implanting said device in vivo and allowing said polymeric shell of said capsules to gradually degrade, thereby resulting in controlled release of said drug.

44. The use as defined in claim 43, wherein said controlled release is step-wise.

45. The use as defined in claim 43, wherein said drug is water insoluble.

46. A method of delivering a drug at a target location comprising:
(a) providing a drug delivery device as defined in claim 8;
(b) delivering said device to said target location; and
(c) allowing said polymeric shell of said capsules to gradually biodegrade at said target location to cause controlled release of said drug from exposed outer portions of said foam.
